(19) United States
(12) Patent Application Publication
(10) Pub. No.: US 2021/0360956 A1
(43) Pub. Date: Nov. 25, 2021

(54) Method for Lengthening Telomeres, Reducing DNA Damage, and Reducing UV-Induced Skin Aging

(71) Applicant: Jacob Rosenstein, Arlington, TX (US)

(72) Inventor: Jacob Rosenstein, Arlington, TX (US)

(21) Appl. No.: 17/107,548

(22) Filed: Nov. 30, 2020

Related U.S. Application Data
(62) Division of application No. 16/195,735, filed on Nov. 19, 2018, now abandoned.
(60) Provisional application No. 62/588,260, filed on Nov. 17, 2017.

(51) Int. Cl. A23L 33/105; A23L 33/16; A23L 33/155

(52) U.S. Cl. CPC A23L 33/105; A23L 33/155; A23L 33/16

(57) ABSTRACT

A method for lengthening telomeres, for countering oxidative damage to DNA caused by free radicals, and for reducing UV-induced skin aging, the method comprising combining nutrients, in at least one of a capsule, tablet, softgel, powder, or liquid form, 20 mcg-20 mg astaxanthin/day with at least one of 100-5,000 IU vitamin D/day, 1-100 mg zinc/day, 1-500 mg niacinamide/day, 1-1,000 IU vitamin E/day, 1 mcg-100 mg lycopene/day, 1 mcg-100 mg beta-carotene/day, 1 mcg-500 mcg selenium/day, 1 mcg-100 mg lutein/day, and 1 mcg-100 mg zeaxanthin/day.

### CROSS-REFERENCE TO RELATED APPLICATIONS

This application is a divisional application of prior application Ser. No. 16/195,735, filed Nov. 19, 2018, which application claims the benefit of U.S. Provisional Application No. 62/588,260, filed Nov. 17, 2017, which applications are hereby incorporated herein by reference in their entirety.

### TECHNICAL FIELD

The invention relates generally to dietary supplements and, more particularly, to a method of using dietary supplements comprising nutrients for lengthening telomeres, reducing DNA damage, and reducing UV-induced skin aging.

### BACKGROUND

There are currently ten primary theories on aging which may be classified under the general headings of programmed aging or unprogrammed aging. These include programmed cell death, telomere theory, gene theory, gene mutation theory, cross-linkage theory, free radical theory, stress protein theory, cellular garbage or accumulated waste theory, wear and tear theory, and autoimmune theory.

To a large extent, the term ‘programmed aging’ may be described as aging resulting from genetically programmed factors. In BIOLOGY OF HUMAN AGING, 2^{nd} Ed., page 17, Prentice Hall, 1999, Alexander P. Spence indicated that a strong argument can be made for some manner of programmed aging since aging begins at birth and each species seems to have its own average lifespan. Chief among the theories contributing to the premise of programmed aging is the telomere theory of aging.

The telomere theory of aging focuses on a cell's inability to successively replicate DNA without eventually placing chromosomes at risk and preventing further cell replication. This theory has been described by Tracy M. Bryan and Roger R. Reddel in “Telomerase, Immortality and Cancer”, published in Today's Life Science, January 1996, pages 26-28, as a situation where human somatic cells lose a certain number of base pairs of DNA, known as telomeres, from the end of each chromosome every time cell division takes place. This is a result of the fact that DNA polymerase, which builds corresponding DNA strands, cannot start the synthesis of the new strand, but must wait for primase to do the job. As a result, the telomere section of the chromosome cannot be reproduced. Although the telomeres are ‘sacrificial’ DNA without any necessary information content, there is still a problem. When these telomeres have decreased to a critical length, cell division ceases; although cell senescence may continue for a time.

Perhaps the best evidence for this theory is the discovery that cultured normal human and animal cells have a finite ability to replicate. Fibroblast cells taken from adults would only divide about 20 times in vitro. However, according to Spence, this limit is rarely, if ever, reached in the body. This finite ability to replicate is known as the “Hayflick limit”.

To some extent, the process of telomere shortening is slowed by the enzyme telomerase, whose purpose it is to add telomere length to DNA. Telomerase is found in some cells (e.g., germ cells and stem cells), which must divide continually to perform their functions. By promoting telomerase activity, it is possible to increase telomere length and consequently extend the period of time that cellular division can take place. While this will not make cells immortal, it may extend their lifespan.

“Unprogrammed aging” may be described as aging resulting from molecular damage to normal body cells and molecules. Chief among the theories contributing to the premise of unprogrammed aging is the free radical theory of aging.

Spence describes free radicals as molecules with unpaired electrons, formed as a result of normal interaction with oxygen. Free radicals react chemically with other molecules, causing oxidative damage. The free radical theory of aging is described as free radical damage to macromolecules such as lipids, proteins and DNA, which may initiate changes explaining the various unprogrammed theories of aging.

Conversely, endogenous (produced by the body) and exogenous (obtained from food or supplements) antioxidants act as free radical scavengers, preventing and repairing damages caused by Reactive Oxygen Species (ROS) and Reactive Nitrogen Species (RNS), and therefore can enhance immune defenses and lower the risk of degenerative diseases.

An understanding of the aforementioned theories of aging has value to the extent that an approach can be developed to promote a process of healthy aging. Since the most obvious manifestation of aging is the physical appearance of skin—namely, fine lines and wrinkles as well as skin density and thickness, an approach to healthy aging should be inclusive of addressing the appearance of aging skin.

Currently, there is a lack of practical strategies for addressing telomere shortening, although there is some research suggesting that supplementation with a common multivitamin may have some value for this purpose. By contrast, there are a great many antioxidants available in supplemental form to help reduce and counter oxidative damage caused by free radicals. In addition, there are topical sunscreen products that help reduce UV-induced skin aging. There are, however, limitations associated with the use of common multivitamins, antioxidants, and topical sunscreens, some of which limitations are discussed as follows:

Multivitamin: In a cross-sectional analysis of data from 586 participants (age 35-74 years) published in the American Journal of Clinical Nutrition (2009; 89(6):1857-63), common multivitamin use and nutrient intakes were assessed with a 146-item food-frequency questionnaire, and relative telomere length of leukocyte DNA was measured. The results were that after age and other potential confounders were adjusted for, multivitamin use was associated with longer telomeres. Compared with nonusers, the relative telomere length of leukocyte DNA was on average 5.1% longer among daily multivitamin users (P for trend=0.002). While not without value, the limitation of this study is that the multivitamins used were relatively low potency, and the study did not address the issue of DNA damage due to oxidation.

Antioxidants: The spectrum of antioxidants available in supplemental form is extensive. That being said, specific antioxidants tend to address specific free radicals, as demonstrated in Oxygen Radical Absorbance Capacity (ORAC) tests. Additionally, specific antioxidants have been shown to provide some specific benefits with regard to disease prevention and treatment, such as alpha lipoic acid for insulin sensitivity in diabetes. While such antioxidants certainly make a valuable contribution to human health and wellness, they have not necessarily been shown to address the issue of DNA damage due to oxidation, nor do they always address other signs of aging

Sunscreen: While topical application of sunscreen certainly helps reduce UV-induced skin aging, it has been noted that it may also have limited efficacy due to inadequacy of application to the skin or removal by perspiring.

Given the aforementioned limitations of common multivitamins, antioxidants and topical sunscreens, a continuing search has been directed to the development of a more effective approach to (1) address telomere shortening, (2) counter oxidative damage to DNA caused by free radicals, and (3) reduce UV-induced skin aging.

### SUMMARY

The present invention, accordingly, provides a method for lengthening telomeres, for countering oxidative damage to DNA caused by free radicals, and for reducing UV-induced skin aging. The method preferably comprises combining nutrients, including 20 mcg-20 mg astaxanthin/day with one of 100-5,000 IU vitamin D/day, 1-100 mg zinc/day, 1-500 mg niacinamide/day, 1-1,000 IU vitamin E/day, 1 mcg-100 mg lycopene/day, 1 mcg-100 mg beta-carotene/day, 1 mcg-500 mcg selenium/day, 1 mcg-100 mg lutein/day, and 1 mcg-100 mg zeaxanthin/day.

In a further embodiment of the invention, the nutrients are combined in at least one of a capsule, tablet, softgel, powder, or liquid form.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### DETAILED DESCRIPTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not intended to be limited to the embodiments shown, but is to be accorded the widest scope consistent with the principles and features disclosed herein. Additionally, as used herein, the term “substantially” is to be construed as a term of approximation. Still further, where ranges are given, they are intended to include all ranges of more narrow scope within the bounds of any respective range given.

In accordance with principles of the present invention, compositions and associated methods of use are exemplified which are effective for helping to reduce telomere shortening, helping to counter oxidative damage to DNA caused by free radicals, and helping to reduce UV-induced skin aging. Such compositions include combining, by way of any suitable method or process, astaxanthin with one or more of vitamin D, zinc, niacinamide, antioxidants (a combination of vitamin E, lycopene, beta-carotene and selenium), and carotenoids (a combination of lutein, zeaxanthin and astaxanthin), as discussed in further detail below.

Astaxanthin has been demonstrated in in vitro studies to improve the function of mitochondria and has good protective effects on human fibroblasts. In that way, it can protect skin cells from free radicals and preserve the collagen layer which result in smooth and youthful appearance of the skin. In the following combinations of ingredients, the quantity of astaxanthin is preferably 20 mcg-20 mg/day, though the range could vary; for example, the range could be 50 mcg-15 mg/day, 100 mcg-10 mg/day, 500 mcg-5 mg/day, or 1 mg-3 mg/day. It is contemplated that the astaxanthin may be in the form of a capsule, tablet, softgel, powder, or liquid.

In one preferred embodiment, the aforementioned amount of astaxanthin may be combined with 100-5,000 IU vitamin D/day in capsule, tablet, softgel, powder, or liquid form for the purpose of helping to address telomere shortening, helping to counter oxidative damage to DNA caused by free radicals, and helping to reduce UV-induced skin aging. Vitamin D is known to be a potent inhibitor of the proinflammatory response and thereby diminishes turnover of leukocytes. Leukocyte telomere length (LTL) is a predictor of aging-related disease and decreases with each cell cycle and increased inflammation. It is contemplated that the range of Vitamin D may vary; for example, the range could be 250-4,000 IU Vitamin D/day, 500-3,000 IU Vitamin D/day, 750-2,000 IU Vitamin D/day, or 1,000-1,100 IU Vitamin D/day.

In an alternative preferred embodiment, the aforementioned amount of astaxanthin may be combined with 1-100 mg zinc/day in capsule, tablet, softgel, powder, or liquid form for the purpose of helping to address telomere shortening, helping to counter oxidative damage to DNA caused by free radicals, and helping to reduce UV-induced skin aging. A possible mechanism for zinc's effects was seen in an in vitro study on rat cell line where low intracellular zinc resulting from nutritional deficiencies induced oxidative DNA damage, and disrupted p53, NF-κB and AP-1 DNA binding that in turn affect DNA repair. It is contemplated that the range of zinc may vary; for example, the range could be 5-80 mg zinc/day, 10-70 mg zinc/day, 20-60 mg zinc/day, or 30-50 mg zinc/day.

In a further alternative preferred embodiment, the aforementioned amount of astaxanthin may be combined with 1-500 mg niacinamide/day in capsule, tablet, softgel, powder, or liquid form for the purpose of helping to address telomere shortening, helping to counter oxidative damage to DNA caused by free radicals, and helping to reduce UV-induced skin aging. Niacinamide is a direct metabolic nutritional precursor to the formation of cellular nicotinamide adenine dinucleotide (NAD), which in turn is essential to energy production and is also a co-substrate for the participation of poly (ADP-ribose) polymerase (PARP) in DNA repair. It is contemplated that the range of niacinamide may vary; for example, the range could be 10-400 mg niacinamide/day, 20-300 mg niacinamide/day, 30-200 mg niacinamide/day, or 50-100 mg niacinamide/day.

In a still further alternative preferred embodiment, the aforementioned amount of astaxanthin may be combined with 1-1,000 IU Vitamin E/day in capsule, tablet, softgel, powder, or liquid form for the purpose of helping to address telomere shortening, helping to counter oxidative damage to DNA caused by free radicals, and helping to reduce UV-induced skin aging. Vitamin E is a chain-breaking antioxidant that prevents the formation of free radicals. Vitamin Es therapeutic benefits have primarily been attributed to its antioxidant effects. It is contemplated that the range of Vitamin E may vary; for example, the range could be 25-900 IU Vitamin E/day, 100-750 IU Vitamin E/day, 200-500 IU Vitamin E/day, or 300-400 IU Vitamin E/day.

In a still further alternative preferred embodiment, the aforementioned amount of astaxanthin may be combined with 1 mcg-100 mg lycopene/day in capsule, tablet, softgel, powder, or liquid form for the purpose of helping to address telomere shortening, helping to counter oxidative damage to DNA caused by free radicals, and helping to reduce UV-induced skin aging. Lycopene has the most potent antioxidant activity of any common carotenoid, is highly lipophilic and is commonly found within cellular membranes. It scavenges free radicals and quenches singlet oxygen, which prevents oxidative damage to DNA. It is contemplated that the range of lycopene may vary; for example, the range could be 10 mcg-90 mg lycopene/day, 20 mcg-80 mg lycopene/day, 30 mcg-70 mg lycopene/day, or 40 mcg-60 mg lycopene/day.

In a still further alternative preferred embodiment, the aforementioned amount of astaxanthin may be combined with 1 mcg-100 mg beta-carotene/day in capsule, tablet, softgel, powder, or liquid form for the purpose of helping to address telomere shortening, helping to counter oxidative damage to DNA caused by free radicals, and helping to reduce UV-induced skin aging. Beta-carotene has antioxidant activities and prevents lipid peroxidation. Serum beta-carotene levels seem to be inversely related to C-reactive protein levels and the white blood count, markers of inflammation. It is contemplated that the range of beta-carotene may vary; for example, the range could be 10 mcg-50 mg beta-carotene/day, 100 mcg-25 mg beta-carotene/day, 1 mg-10 mg beta-carotene/day, or 2 mg-5 mg beta-carotene/day.

In a still further alternative preferred embodiment, the aforementioned amount of astaxanthin may be combined with 1 mcg-500 mcg selenium/day in capsule, tablet, softgel, powder, or liquid form for the purpose of helping to address telomere shortening, helping to counter oxidative damage to DNA caused by free radicals, and helping to reduce UV-induced skin aging. As selenocysteine, selenium is incorporated into a number of selenoproteins (selenium-dependent antioxidant enzymes). It is these selenoproteins that are responsible for selenium's biological functions. These selenoproteins protect against free radicals and other damaging reactive oxygen species, and help to convert vitamin E and vitamin C from their oxidized forms back into their non-oxidized/antioxidant forms. Selenium also works with copper and zinc to produce the antioxidant enzyme superoxide dismutase, and with iron to produce catalase. It is contemplated that the range of selenium may vary; for example, the range could be 10 mcg-400 mcg selenium/day, 20 mcg-300 mcg selenium/day, 30 mcg-200 mcg selenium/day, or 50 mcg-100 mcg selenium/day.

In a still further alternative preferred embodiment, the aforementioned amount of astaxanthin may be combined with 1 mcg-100 mg lutein/day in capsule, tablet, softgel, powder, or liquid form for the purpose of helping to address telomere shortening, helping to counter oxidative damage to DNA caused by free radicals, and helping to reduce UV-induced skin aging. Lutein is a macular pigment that helps ameliorate light and oxygen damage, and prevents age-related cellular and tissue deterioration in the eye. It also inhibits the activation of NF-κB and the subsequent inhibition of pro-inflammatory mediators. It is contemplated that the range of lutein may vary; for example, the range could be 10 mcg-50 mg lutein/day, 50 mcg-10 mg lutein/day, 100 mcg-5 mg lutein/day, or 1 mg-3 mg lutein/day.

In a still further alternative preferred embodiment, the aforementioned amount of astaxanthin may be combined with 1 mcg-100 mg zeaxanthin/day in capsule, tablet, softgel, powder, or liquid form for the purpose of helping to address telomere shortening, helping to counter oxidative damage to DNA caused by free radicals, and helping to reduce UV-induced skin aging. Like lutein, zeaxanthin is a macular pigment that helps ameliorate light and oxygen damage, and prevents age-related cellular and tissue deterioration in the eye. It also inhibits the activation of NF-κB and the subsequent inhibition of pro-inflammatory mediators. It is contemplated that the range of zeaxanthin may vary; for example, the range could be 10 mcg-50 mg zeaxanthin/day, 50 mcg-25 mg zeaxanthin/day, 100 mcg-10 mg zeaxanthin/day, or 1 mg-5 mg zeaxanthin/day.

It is understood that the present invention may take many forms and embodiments. Accordingly, several variations may be made in the foregoing without departing from the spirit or the scope of the invention.

Having thus described the present invention by reference to certain of its preferred embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features. Many such variations and modifications may be considered obvious and desirable by those skilled in the art based upon a review of the foregoing description of preferred embodiments. Accordingly, it is appropriate that the appended claims be construed broadly and in a manner consistent with the scope of the invention.

1. A method for inhibiting the shortening of telomeres and for lengthening telomeres, for countering oxidative damage to DNA caused by free radicals, and for reducing UV-induced skin aging, the method comprising combining 20 mcg-20 mg astaxanthin/day with one or more of the following nutrients:
100-5,000 IU vitamin D/day,
1-100 mg zinc/day,
1-500 mg niacinamide/day,
1-1,000 IU vitamin E/day,
1 mcg-100 mg lycopene/day,
1 mcg-100 mg beta-carotene/day,
1 mcg-500 mcg selenium/day,
1 mcg-100 mg lutein/day, and
1 mcg-100 mg zeaxanthin/day.

2. The method of claim 1, wherein the astaxanthin and nutrients are combined in the form of a capsule.

3. The method of claim 1, wherein the astaxanthin and nutrients are combined in the form of a tablet.

4. The method of claim 1, wherein the astaxanthin and nutrients are combined in the form of a softgel.

5. The method of claim 1, wherein the astaxanthin and nutrients are combined in the form of a powder.

6. The method of claim 1, wherein the astaxanthin and nutrients are combined in the form of a liquid.

7. A method for inhibiting the shortening of telomeres and for lengthening telomeres, for countering oxidative damage to DNA caused by free radicals, and for reducing UV-induced skin aging, the method comprising combining 20 mcg-20 mg astaxanthin/day with the following nutrients:
100-5,000 IU vitamin D/day,
1-100 mg zinc/day,
1-500 mg niacinamide/day,
1-1,000 IU vitamin E/day,
1 mcg-100 mg lycopene/day,
1 mcg-100 mg beta-carotene/day,
1 mcg-500 mcg selenium/day,
1 mcg-100 mg lutein/day, and
1 mcg-100 mg zeaxanthin/day.

8. The method of claim 7, wherein the astaxanthin and nutrients are combined in the form of a capsule.

9. The method of claim 7, wherein the astaxanthin and nutrients are combined in the form of a tablet.

10. The method of claim 7, wherein the astaxanthin and nutrients are combined in the form of a softgel.

11. The method of claim 7, wherein the astaxanthin and nutrients are combined in the form of a powder.

12. The method of claim 7, wherein the astaxanthin and nutrients are combined in the form of a liquid.

13. A method for inhibiting the shortening of telomeres and for lengthening telomeres, for countering oxidative damage to DNA caused by free radicals, and for reducing UV-induced skin aging, the method comprising steps of:
encapsulating in capsules 10 mcg-10 mg astaxanthin in powder form with the following nutrients in powder form:
about 2,000 IU vitamin D,
about 20 mg zinc,
about 100 mg niacinamide,
about 20 mg vitamin E,
about 6 mg lycopene,
about 1,500 mcg beta-carotene,
about 100 mcg selenium,
about 10 mg lutein, and
about 2 mg zeaxanthin; and
administering two capsules per day to a consumer.
